(19) United States
(12) Patent Application Publication
(10) Pub. No.: US 2013/0296682 A1
(43) Pub. Date: Nov. 7, 2013

(54) INTEGRATING PRE-SURGICAL AND SURGICAL IMAGES

(71) Applicants: John Clavin, Seattle, WA (US); Jaron Lanier, Berkeley, CA (US)

(72) Inventors: John Clavin, Seattle, WA (US); Jaron Lanier, Berkeley, CA (US)

(73) Assignee: MICROSOFT CORPORATION, Redmond, WA (US)

(21) Appl. No.: 13/464,750

(22) Filed: May 4, 2012

(51) Int. Cl. A61B 1/04; A61B 6/00

(52) U.S. Cl. 600/407; 600/109

(57) ABSTRACT

Embodiments are disclosed that relate to the integration of pre-surgical images and surgical images. For example, one disclosed embodiment provides, on a computing system, a method including receiving a pre-surgical image of a patient, receiving a depth image of the patient during surgery, and comparing the depth image of the patient to the pre-surgical image of the patient. The method further comprises providing an output based upon a result of comparing the depth image of the patient to the pre-surgical image of the patient.

### BACKGROUND

A wide range of imaging technologies are currently used to aid surgeons in diagnosing conditions and performing procedures. For example, a patient may undergo one or more of a sonogram, an X-Ray Computed Tomography (CT) scan, a Positron Emission Tomography (PET) scan, a Magnetic Resonance Imaging (MRI) scan, or other imaging scan, to form an image of a portion of the body prior to a surgical procedure. Such images may help with diagnosis and procedure planning, and also serve as an important reference tool during surgery. Likewise, real-time images may be acquired via an endoscope during surgery. Such images may allow for the use of less invasive surgical techniques.

Medical imaging of different types may be integrated via various systems, including but not limited to picture archiving and communications systems. Such systems coordinate storage and access for images of different types, and may allow different types of image scans to be viewed simultaneously for comparison. Such systems also may be used to deliver pre-surgical images to the operating room for viewing by a surgeon along with real-time endoscopic images. Additionally, image integration systems may be used in combination with picture archiving and communication systems and real-time image acquisition systems (e.g. endoscopes) to allow real-time and archived images to be independently displayed and controlled in an operating room.

### SUMMARY

Embodiments are disclosed that relate to the integration of pre-surgical images and surgical depth images. For example, one disclosed embodiment provides, on a computing system, a method comprising receiving a pre-surgical image of a patient, receiving a depth image of the patient during surgery, and comparing the depth image of the patient to the pre-surgical image of the patient. The method further comprises providing an output based upon a result of comparing the depth image of the patient to the pre-surgical image of the patient.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows an example embodiment of a use environment for combining a pre-surgical image with a depth image acquired during surgery.

FIG. 2 shows example embodiment of a pipeline for processing and integrating pre-surgical images and depth images acquired during surgery.

FIG. 3 shows a flow diagram depicting an embodiment of a method for integrating pre-surgical and surgical images.

FIG. 4 shows a block diagram depicting an example embodiment of a computing device.

### DETAILED DESCRIPTION

Embodiments are disclosed herein that relate to integrating pre-surgical imaging and surgical depth imaging. Such integration may facilitate live viewing and comparison of pre-surgical images and real-time depth images acquired during surgery, Further, as discussed in more detail below, in some embodiments the pre-surgical images and real-time depth images may be converted to a common format that facilitates the automated comparison of the images. This may allow for such scenarios as the generation of automatic alerts for a surgeon. Any suitable type of pre-surgical and surgical depth images may be integrated according to the present disclosure. Examples of pre-surgical imaging technologies include, but are not limited to, CT imaging, PET imaging, MRI imaging, x-ray imaging, ultrasonic imaging, and depth camera imaging. Examples of suitable surgical imaging methods include, but are not limited to, depth imaging via an endoscope and/or a depth camera located external to a patient, as well as color and/or grayscale two-dimensional imaging via an endoscope and/or a camera located external to a patient.

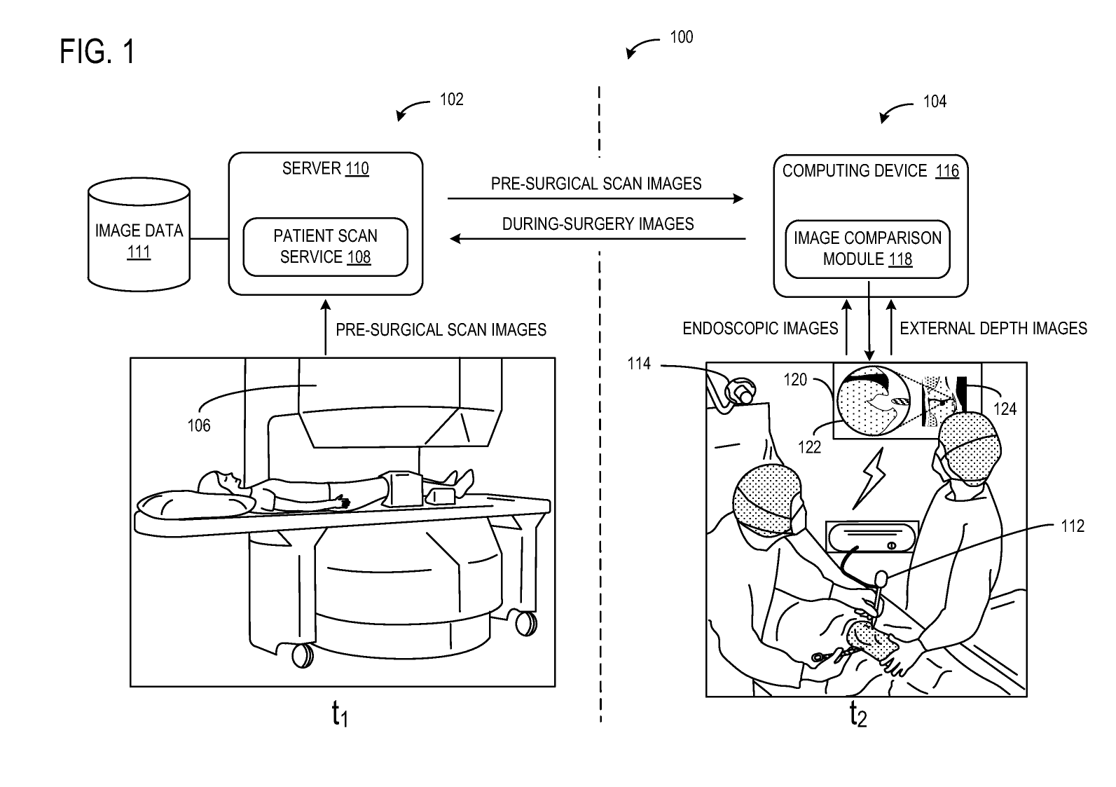

FIG. 1 shows an embodiment of an example use environment 100 for integrating pre-surgical imaging and surgical imaging. Use environment 100 comprises a pre-surgical imaging location 102 and a surgical location 104. The pre-surgical imaging location 102 comprises an imaging device 106 configured to acquire image data of an internal anatomical structure of at least a part of a patient, here illustrated as occurring at a time t₁. Images from the imaging device 106 may be sent to a patient imaging service 108 operating on a computing system 110. The images may then be stored in an image data store 111.

The surgical location 104 comprises one or more imaging devices configured to capture a depth image of a patient during a surgical procedure, here illustrated at occurring at a time t₂ that is later than t₁. For example, the surgical location 104 may comprise an endoscope 112 comprising a depth camera and/or a color/grayscale two-dimensional camera, and/or may comprise an external depth camera 114. The surgical location also may comprise an external two-dimensional camera, as well as microphones, speakers, and any other suitable input and output devices. The term “endoscope” as used herein represents any instrument that may be inserted into a patient's body to allow optical imaging of internal structures of the patient's body.

Image data acquired during surgery may be provided to a computing device 116, and also may be sent to the patient imaging service 108 for storage in the image data store 111. Likewise, pre-surgical image data also may be provided to the computing device 116 by the patient imaging service 108 during surgery for comparison to the images acquired during surgery, e.g. via an image comparison module 118. The image comparison module 118 may then provide output to a display device 120 for display of information related to the comparison and/or integration of the pre-surgical images and surgical images.

In some embodiments, the pre-surgical images and the surgical images may have a shared format that facilitates comparing the images. Further, the pre-surgical images and surgical images may be processed, e.g. via classification, to identify structures in the images. Various statistical image-based classification methods may allow images of healthy anatomical structures to be distinguished from unhealthy structures (e.g. diseased, malignant, torn/broken/ruptured, etc.), and therefore may aid in the identification of conditions by endoscopic imaging. Functions for performing such classifications may be trained using a training set of image data comprising a variety of different tissue conditions for each anatomical structure of interest to allow conditions of anatomical structures to be distinguished. It will be understood that such processing may occur at the time of image acquisition and storage (e.g. prior to surgery for the pre-surgical images), and/or may be performed at the time of surgery.

In some embodiments, a physician also may input information regarding the condition of the patient for storage with the pre-surgical images. Such information may include, but is not limited to, information related to the diagnosis of the patient and information related to a surgical procedure to be performed on the patient. With such information, surgical images may be compared with the pre-scan images to determine whether the observed surgical images match expected surgical images based upon the patient condition information. Further, notifications may be generated and output based upon such comparisons. For example, in some embodiments, external depth images may be compared to such data used to determine whether a surgery is performed on a correct body part or side. As a more specific example, external depth images acquired during surgery may be analyzed to determine that a surgeon is operating on a particular limb of a patient. Such information may be compared to condition-related information to determine whether the procedure is being performed on the correct limb.

Further, a notification of an identity of an anatomical structure and/or condition may be provided as an output during surgery based upon a comparison of the pre-surgical images to the surgical images. For example, a classification function may be trained to classify anatomical structures based upon appearance, location (e.g. within a three-dimensional map of the body), and any other suitable information. In some embodiments, such a function may be trained for a particular surgery being performed, such that images for a selected type of surgery will be classified based upon a function trained via a training set of image data from the same surgical procedure. In other embodiments, such a function may not be limited to images expected during a particular surgical procedure, but instead may be trained on a wide range of anatomical images. In such embodiments, external depth images from the surgical location may be used as a filter to limit a group of anatomical structures that may be matched to structures in an observed endoscopic image. As an example, if an analysis of endoscopic image data indicates that an observed image may be meniscus cartilage or left ventricle tissue with statistically similar probabilities, an external depth image may be used to determine that the endoscope is located near a patient's left knee, and therefore filters out the possibility of the image being a cardiovascular body part.

Such a recognition system may identify unexpected anomalies not present in a healthy anatomy, such as a tumor, and to generate an alert (e.g. by highlighting in a displayed image) based upon the detected anatomical condition. Any suitable notification may be provided. As one nonlimiting example, a recognition system may be configured to highlight the anomalies in the image displayed on the display device.

In some embodiments, instead of performing a pre-surgical scan that images internal anatomical structures, a pre-surgical scan may be performed that images a patient's external body. For example, a patient may be scanned with a depth camera. In such embodiments, the depth camera scan of the patient may be used to generate a virtual representation of an internal anatomy of the patient, for example, by fitting the actual shape of the patient's body to a model virtual internal anatomy, such that fitting the overall shape of the model to the patient results in fitting of the model internal anatomy to the patient. Such a virtual anatomy may then be used during surgery in the manner described above for other pre-surgical image data.

The external depth camera 114 also may be utilized to determine a pose of the patient during surgery, and adjust a presentation of a pre-surgical image based upon a pose so that the orientation of the pre-surgical image as displayed to the surgeon matches that of the body part on which the surgeon is operating. Referring to FIG. 1, the display device 120 is shown as displaying an endoscopic image 122, and also displaying a pre-surgical image 124 that illustrates a location at which the endoscopic image is taken. As depicted, the orientation and pose of the pre-surgical image of the patient's knee joint image matches the orientation and pose of the knee joint from the surgeon's perspective in the operating room. In this manner, images from a pre-surgical scan of the patient may be applied to an operating room of a patient without having to place any reference markings or devices on the patient. It will be understood that any adjustments made to the patient's pose in the operating room as detected by the external depth camera 114 may automatically update the pose and orientation applied to the pre-surgical image.

Where the endoscope 112 includes a depth camera, images from the endoscopic depth camera may be used to create a three-dimensional image of the subject anatomical area. This image may then be output to the display device 120 to give the surgeon a detailed live depth-enhanced view of the subject anatomical area. Likewise, the live image from the endoscope 112 also may include color and/or grayscale images from a two-dimensional image sensor. In some embodiments, a surgeon may manipulate such images on the display device 120 via voice commands and/or gesture commands to preserve a sterile environment while seeking a desired view of the patient's anatomical images.

Further, depth images acquired via an endoscope with a depth sensor may be stored for comparison with later-received endoscope images and/or later internal anatomical scans (e.g. CT, PET, MRI, x-ray, etc.), to identify and highlight any changes over time. As a more specific example, an endoscope may be used to locate and map a tumor for comparison to later scan images after a course of treatment to measure any changes in tumor size.

In some embodiments, augmented reality (AR) techniques may be used in combination with the above-described methods, for example, to help predict the outcome of a procedure. As one example, an external depth image of a patient may be used as a model for demonstrating potential outcomes of plastic surgery by adding shape-mapped and scale-mapped (and potentially texture-mapped) images over the depth image of the user's face or other body part. Likewise, AR techniques also may be used during a surgical procedure, for example, to observe how potential stitches (displayed via AR techniques applied to endoscopic images) may interact with other anatomy, and to choose stitch locations based upon such AR demonstrations. It will be understood that these specific scenarios are presented for the purpose of example, and are not intended to be limiting in any manner.

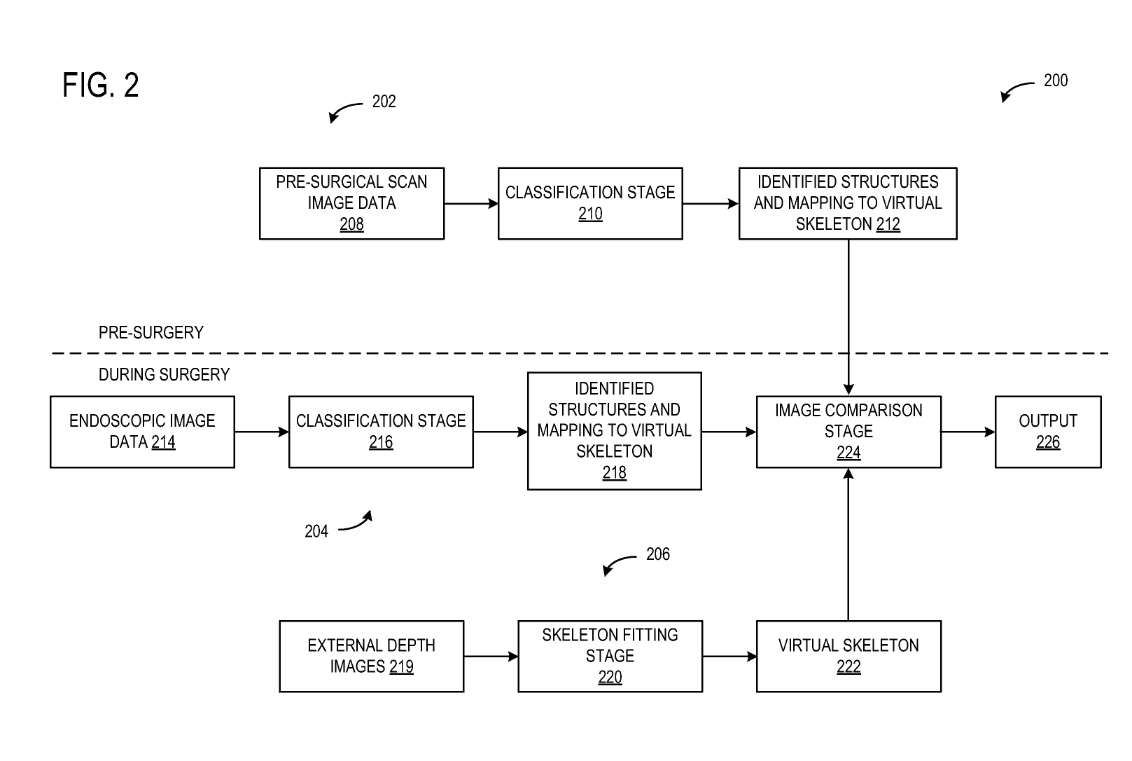

As mentioned above, the use of a complementary image format for pre-surgical images and surgical depth images may facilitate comparison of the images. FIG. 2 shows an example embodiment of a processing pipeline 200 to convert such data into a common format. Processing pipeline 200 comprises a pre-surgical image processing pipeline 202, a surgical endoscopic image processing pipeline 204, and a surgical external depth image pipeline 206.

First referring to the pre-surgical image processing pipeline 202, the images 208 acquired in a pre-surgical image scan may be processed via a classification stage 210. The classification stage 210 may utilize, for example, a classification function that has been trained based upon a training set of scan data that corresponds to a variety of anatomical structures and associated conditions. The classification stage 210 may output identifications 212 of anatomical structures captured in the pre-surgical images 208, and also may output a mapping of the structures to a virtual skeletal model that may be used to associate different image types. As mentioned above, the identifications of the anatomical structures may comprise information regarding one or more conditions of the anatomical structures.

Next referring to the endoscopic image processing pipeline 204, endoscopic images 214 also may be processed via a classification stage 216. As described above, such a classification stage 216 may utilize, for example, a classification function that has been trained based upon a training set of endoscopic image data that corresponds to a variety of anatomical structures and associated conditions. In some embodiments, such classification functions may be specific to surgical techniques, such that different functions are used to classify image data for different procedures, while in other embodiments such functions may be more global. Further, different functions may be used for depth image data and two-dimensional image data. The classification stage 216 may output identifications 218 of anatomical structures captured in the endoscopic images 214, and also may output a mapping of the structures to a virtual skeletal model.

Referring next to external depth image processing pipeline 206, a skeleton fitting process 220 may be applied to an external depth image 219 to produce a virtual skeleton 222 to thereby provide a machine readable representation of the patient. In other words, the virtual skeleton 222 is derived from the external depth image 219 to model the patient, or a portion of the patient. It will be understood that any suitable skeletal fitting algorithms may be applied to the external depth image 219 to produce the virtual skeleton 222.

The virtual skeleton 222 may include a plurality of joints, and each joint may correspond to a joint of the patient, or to any other suitable portion of the patient (e.g. one virtual skeleton joint may represent plural real joints, such as vertebral joints). Each joint may be associated with any suitable parameters (e.g., three dimensional joint position, joint rotation, body posture of corresponding body part (e.g., hand open, hand closed, etc.) etc.). It is to be understood that a virtual skeleton 222 may take the form of a data structure including one or more parameters for each of a plurality of skeletal joints (e.g., a joint matrix including an x position, a y position, a z position, and a rotation for each joint). In some embodiments, other types of virtual skeletons may be used (e.g., a wireframe, a set of shape primitives, etc.). Further, a mapping of other anatomical structures to such a virtual skeleton may be based upon a relative location of such structures to the joints of the virtual skeleton, upon attachment points of the other anatomical structures to the virtual skeleton, and/or upon any other suitable relationship.

The output from each of the image processing pipelines of FIG. 2 may be provided to an image comparison stage, as shown at 224. The image comparison stage 224 may be configured to compare the output of the pre-surgical image processing pipeline (e.g. identifications 212) with the outputs of the endoscopic image processing pipeline (e.g. identifications 218) and/or the external depth camera image processing pipeline (e.g. virtual skeleton 222). With these inputs, the image comparison stage 224 may then compare the pre-surgical information with the surgical image information, as described above, to produce an output 226. Any suitable output 226 may be provided. For example, the image comparison stage 224 may compare the pre-surgical and surgical data to form a combined image that shows pre-surgical image data in combination with surgical image data. One nonlimiting example of such a combined image is shown in FIG. 1, in which a live endoscopic image is shown along with a pre-surgical image indicating the location at which the endoscopic image is acquired. In another example, a portion of the pre-surgical image corresponding to the field of view of the endoscopic image may be superimposed over the endoscopic image to help highlight a location of tissue damage or other condition. Such combined images may represent a pose and/or an orientation as determined from a comparison between a pre-surgical image and surgical external depth images.

Another example of an output 226 that may be provided by image comparison stage 224 comprises an anatomical avatar. Such an avatar may be produced, for example, by displaying a model anatomical avatar fitted to the virtual skeleton, along with anatomical structures identified in the pre-surgical images in a location based upon the mappings of the structures to the virtual skeleton. As a more specific example, a knee joint ligament imaged in an MRI scan may be displayed on a virtual anatomical avatar based upon expected attachment points of the ligament to bones of the knee joint, or upon observed attachment points from the MRI scan.

Yet another example of an output that may be provided comprises a notification. Examples of notifications may include, but are not limited to, an alert that an observed anatomical structure does not match an expected anatomical structure, and that a location of the patient on which surgery is being performed matches an expected location. Such notifications may be output to a display device, audibly via a speaker in the operating room, or in any other suitable manner. It will be understood that the above-described examples of outputs 226 are presented for the purpose of example, and are not intended to be limiting in any manner.

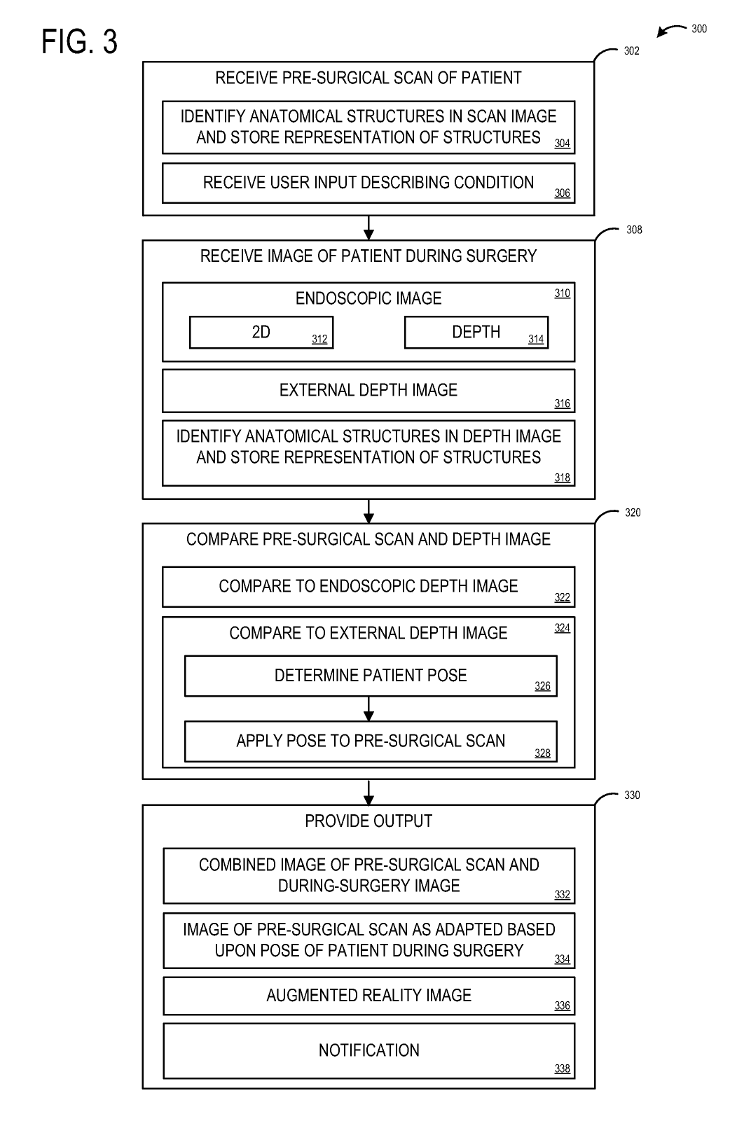

FIG. 3 shows a flow diagram depicting an embodiment of a method 300 for integrating pre-surgical and surgical images. Method 300 comprises, at 302, receiving a pre-surgical image of a patient. Receiving the pre-surgical image may comprise, at 304, identifying anatomical structures captured in the image to identify the anatomical structures, and storing the image and representations of the anatomical structures. Any suitable representations of the anatomical structures may be stored, including but not limited to an identification of the anatomical structures and potentially one or more conditions thereof, as well as a mapping of anatomical structures to a model anatomy, such as a virtual skeleton. Further, the anatomical structures may be identified in any suitable manner, including but not limited to via a classification process utilizing a trained classification function. It will be understood that the processed pre-surgical may be stored in a format compatible with those in which surgical images are stored. Receiving the pre-surgical image also may comprise, at 306, receiving an input describing a condition of the patient. For example, where a patient has a torn medial collateral ligament, such information may specify which knee has this condition, as well as other details about the injury.

Method 300 next comprises, at 308, receiving an image of the patient during surgery. As described above, such a depth image may comprise an endoscopic image 310, which may include a two-dimensional image 312 (e.g. RGB data) and/or depth image 314. The image of the patient received during surgery also may comprise an external depth image 316 from an external depth sensor located in the operating room. Method 300 next comprises identifying anatomical structures captured in the surgical image, and storing the image and representations of the structures identified. As described above, identifying anatomical structures may include fitting a virtual skeleton to an image of the patient, classifying internal anatomical structures of the patient, and mapping identified structures to a virtual skeleton.

Continuing, method 300 comprises, at 320, comparing the pre-surgical image and the surgical depth image. This may comprise, at 322, comparing the pre-surgical image to an endoscopic depth image, and/or, at 324, comparing the pre-surgical image to a surgical external depth image. Where the pre-surgical image is compared to an external depth image, method 300 may comprise, at 326, determining a pose of the patient during surgery, and at 328, applying the pose of the patient during surgery to the pre-surgical image. It will be understood that comparing the pre-surgical image and the surgical depth image may comprise comparing the representations of the anatomical structures identified in each of the images.

Method 300 next comprises, at 330, providing an output based upon the comparison of the pre-surgical image and the surgical image. Any suitable output may be provided. For example, as described above, the method may comprise outputting a combined image of the pre-surgical image and surgical image, as indicated at 332. The output also may comprise an image of pre-surgical image as adapted based upon a pose of patient during surgery, as indicated at 334, or a virtual anatomy as adapted based upon the pose of the patient. The output may further comprise an augmented reality image, as indicated at 336 and as described in more detail above. Additionally, the output may comprise a notification, as indicated at 338, and as described in more detail above.

In some embodiments, the above described methods and processes may be tied to a computing system including one or more computers. Examples of such computing systems may include, but are not limited to, imaging device 106, computing system 110, endoscope 112, depth camera 114, and/or computing device 116 of FIG. 1. In particular, the methods and processes described herein may be implemented as a computer application, computer service, computer API, computer library, and/or other computer program product.

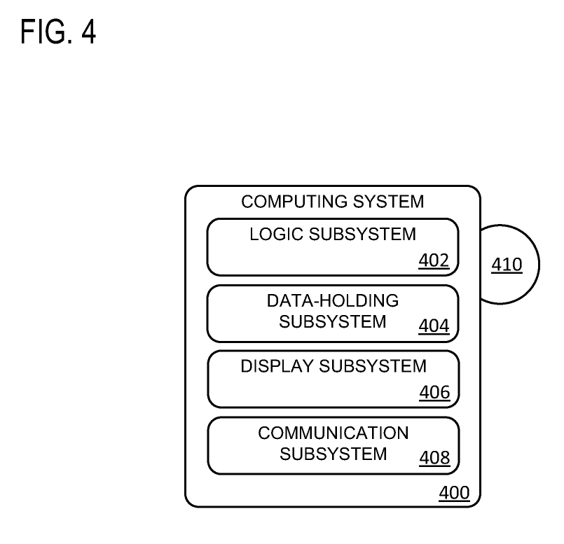

FIG. 4 schematically shows a nonlimiting computing system 400 that may perform one or more of the above described methods and processes. Computing system 400 is shown in simplified form. It is to be understood that virtually any computer architecture may be used without departing from the scope of this disclosure. In different embodiments, computing system 400 may take the form of a mainframe computer, server computer, desktop computer, laptop computer, tablet computer, home entertainment computer, network computing device, mobile computing device, mobile communication device, gaming device, etc.

Computing system 400 includes a logic subsystem 402 and a data-holding subsystem 404. Computing system 400 may optionally include a display subsystem 406, communication subsystem 408, and/or other components not shown in FIG. 4. Computing system 400 may also optionally include user input devices such as keyboards, mice, game controllers, cameras, microphones, and/or touch screens, for example.

Logic subsystem 402 may include one or more physical devices configured to execute one or more instructions. For example, logic subsystem 402 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

Logic subsystem 402 may include one or more processors that are configured to execute software instructions. Additionally or alternatively, logic subsystem 402 may include one or more hardware or firmware logic machines configured to execute hardware or firmware instructions. Processors of logic subsystem 402 may be single core or multicore, and the programs executed thereon may be configured for parallel or distributed processing. Logic subsystem 402 may optionally include individual components that are distributed throughout two or more devices, which may be remotely located and/or configured for coordinated processing. One or more aspects of logic subsystem 402 may be virtualized and executed by remotely accessible networked computing devices configured in a cloud computing configuration.

Data-holding subsystem 404 may include one or more physical, non-transitory, devices configured to hold data and/or instructions executable by logic subsystem 402 to implement the herein described methods and processes. When such methods and processes are implemented, the state of data-holding subsystem 404 may be transformed (e.g., to hold different data).

Data-holding subsystem 404 may include removable media and/or built-in devices. Data-holding subsystem 404 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 404 may include devices with one or more of the following characteristics: volatile, nonvolatile, dynamic, static, read/write, read-only, random access, sequential access, location addressable, file addressable, and content addressable. In some embodiments, logic subsystem 402 and data-holding subsystem 404 may be integrated into one or more common devices, such as an application specific integrated circuit or a system on a chip.

FIG. 4 also shows an aspect of the data-holding subsystem in the form of removable computer-readable storage media 410, which may be used to store and/or transfer data and/or instructions executable to implement the herein described methods and processes. Removable computer-readable storage media 410 may take the form of CDs, DVDs, HD-DVDs, Blu-Ray Discs, EEPROMs, and/or floppy disks, among others.

It is to be appreciated that data-holding subsystem 404 includes one or more physical, non-transitory devices. In contrast, in some embodiments aspects of the instructions described herein may be propagated in a transitory fashion by a pure signal (e.g., an electromagnetic signal, an optical signal, etc.) that is not held by a physical device for at least a finite duration. Furthermore, data and/or other forms of information pertaining to the present disclosure may be propagated by a pure signal.

The terms “module,” “program,” and “engine” may be used to describe an aspect of computing system 400 that is implemented to perform one or more particular functions. In some cases, such a module, program, or engine may be instantiated via logic subsystem 402 executing instructions held by data-holding subsystem 404. It is to be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” are meant to encompass individual or groups of executable files, data files, libraries, drivers, scripts, database records, etc.

It is to be appreciated that a “service,” as used herein, may be an application program executable across multiple user sessions and available to one or more system components, programs, and/or other services. In some implementations, a service may run on a server responsive to a request from a client.

When included, display subsystem 406 may be used to present a visual representation of data held by data-holding subsystem 404. As the herein described methods and processes change the data held by the data-holding subsystem, and thus transform the state of the data-holding subsystem, the state of display subsystem 406 may likewise be transformed to visually represent changes in the underlying data. Display subsystem 406 may include one or more display devices utilizing virtually any type of technology. Such display devices may be combined with logic subsystem 402 and/or data-holding subsystem 404 in a shared enclosure, or such display devices may be peripheral display devices.

When included, communication subsystem 408 may be configured to communicatively couple computing system 400 with one or more other computing devices. Communication subsystem 408 may include wired and/or wireless communication devices compatible with one or more different communication protocols. As nonlimiting examples, the communication subsystem may be configured for communication via a wireless telephone network, a wireless local area network, a wired local area network, a wireless wide area network, a wired wide area network, etc. In some embodiments, the communication subsystem may allow computing system 400 to send and/or receive messages to and/or from other devices via a network such as the Internet.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof.

1. On a computing system, a method comprising:
receiving a pre-surgical image of a patient;
receiving a depth image of the patient during surgery;
comparing the depth image of the patient to the pre-surgical image of the patient; and
outputting a notification based upon a result of comparing the depth image of the patient to the pre-surgical image of the patient.

2. The method of claim 1, further comprising receiving a user input of data describing a condition of the patient and storing the pre-surgical image and the data describing the condition of the patient, and wherein outputting the notification comprises outputting the alert notification based on comparing the depth image of the patient to the data describing the condition of the patient.

3. The method of claim 2, wherein the depth image is an external depth image, and wherein the notification comprises an alert that a location of the surgery on the patient does not match an expected location.

4. The method of claim 1, wherein receiving the depth image of the patient during surgery further comprises classifying one or more internal anatomical structures imaged in the depth image, and storing a representation of the one or more internal anatomical structures imaged in the depth image.

5. The method of claim 4, wherein classifying the one or more internal anatomical structures imaged in the depth image comprises determining a condition of an identified internal anatomical structure.

6. The method of claim 5, wherein outputting the notification comprises outputting a notification regarding the condition.

7. The method of claim 1, wherein the depth image is received via an endoscopic depth camera.

8. The method of claim 7, further comprising receiving two-dimensional image data from an endoscopic two-dimensional image sensor, and outputting the two-dimensional image data to a display device along with the depth image.

9. The method of claim 7, further comprising comparing the depth image to a later-received depth image and providing an output showing a change between the depth image and the later-received depth image.

10. The method of claim 7, wherein the depth image is an endoscopic depth image, and further comprising:
receiving an external depth image from a depth sensor external to the patient, and utilizing the external depth image as a filter in identifying an anatomical structure of the patient via the endoscopic depth image and the pre-surgical image.

11. The method of claim 1, wherein receiving the depth image of the patient during surgery comprises receiving depth image data from a depth camera external to the patient.

12. The method of claim 11, wherein comparing the depth image to the pre-surgical image comprises determining a pose of the patient during surgery and applying the pose to the pre-surgical image, and wherein providing the output comprises outputting to a display device an image of the pre-surgical image as adapted based upon the pose of the patient during surgery.

13. The method of claim 11, wherein comparing the depth image to the pre-surgical image comprises determining a pose of the patient during surgery and applying the pose to the pre-surgical image, and wherein providing the output comprises outputting to a display device an image of a model anatomy as adapted based upon the pose of the patient during surgery.

14. The method of claim 1, wherein the notification comprises an alert that the depth image does not match an expected image based upon the pre-surgical image.

15. A method, comprising:
receiving a pre-surgical image of a patient;
processing the pre-surgical image of the patient to identify an internal anatomical structure imaged in the pre-surgical image;
receiving an endoscopic depth image of the patient during surgery;
processing the endoscopic depth image to identify an internal anatomical structure imaged in the endoscopic depth image;
comparing the representation of the internal anatomical structure imaged in the pre-surgical image with the representation of the internal anatomical structure imaged in the endoscopic depth image; and
providing an output based upon comparing the representation of the internal anatomical structure imaged in the pre-surgical image with the representation of the internal anatomical structure imaged in the endoscopic depth image.

16. The method of claim 15, wherein the output comprises a combined image of the pre-surgical image and the endoscopic image indicating a location of the endoscope within the pre-surgical image.

17. The method of claim 15, wherein the output comprises a notification of a condition of the anatomical structure.

18. A computing system, comprising:
a logic subsystem; and
a data-holding subsystem comprising stored instructions executable by the logic subsystem to:
receive a pre-surgical image of a patient and a representation of an anatomical structure of the patient imaged in the pre-surgical image;
receive depth image data from a depth camera external to the patient;
determine from the depth image data a pose of the patient during surgery;
apply the pose to the pre-surgical image; and
output to a display device an image of the pre-surgical image as adapted based upon the pose of the patient during surgery.

19. The computing device of claim 18, wherein the image of the pre-surgical image as adapted based upon the pose of the patient during surgery comprises an image of the internal anatomical structure illustrated within an avatar.

20. The method of claim 19, further comprising updating the output based upon a change in pose of the patient during surgery.
